(19) United States
(12) Patent Application Publication
(10) Pub. No.: US 2005/0065389 A1
(43) Pub. Date: Mar. 24, 2005

(54) Method of increasing the carbon chain length of olefinic compounds

(76) Inventors: Cornelis Johannes De Bruyn, Secunda (ZA); Ewald Watermeyer De Wet, Paarl (ZA); Jan Mattheus Botha, Sasolburg (ZA); Jan Petrus Karel Reynhardt, Vaalpark (ZA)

Correspondence Address: Finnegan Henderson Farabow;Garrett & Dunner, 1300 I Street NW, Washington, DC, 20005-3315, US

(21) Appl. No.: 10/489,216

(22) PCT Filed: Sep. 11, 2002
(86) PCT No.: PCT/ZA02/00138

(30) Foreign Application Priority Data
Sep. 17, 2001 (ZA) 2001/7636

(51) Int. Cl. C07C 1/00

(52) U.S. Cl. 585/639

(57) ABSTRACT

According to the present invention there is provided a process of increasing the carbon chain length of an olefinic compound comprising the steps of providing a starting olefinic compound and subjecting it to hydroformylation to produce an aldehyde and/or alcohol with an increased carbon chain length compared to the starting olefinic compound. Optionally the aldehyde that may form during the hydroformylation reaction is hydrogenated to convert it to an alcohol which has an increased carbon chain length compared to the starting olefinic compound. The alcohol with the increased carbon chain length is subjected to dehydration to produce an olefinic compound with an increased carbon chain length compared to the starting olefinic compound. The invention also relates to olefinic compounds produced by the process.

### TECHNICAL FIELD

This invention relates to a process of increasing the carbon chain length of olefinic compounds. The invention also relates to olefinic compounds produced by this process.

### BACKGROUND ART

There is a high demand for longer chain α-olefins, especially even numbered α-olefins such as 1-hexene and 1-octene. 1-Hexene and 1-octene are used, amongst others, as co-monomers in polyethylene production where they serve as plasticisers e.g. in the preparation of linear low-density polyethylene.

One method of producing olefins is through an olefin metathesis reaction. A disadvantage of this type of reaction is that it is difficult to control the reaction to produce only one specific olefin, and the majority of olefins produced by this process are internal olefins. Metathesis reactions are accordingly not very suitable for preparing α-olefins such as 1-hexene or 1-octene. One type of metathesis reaction, namely ethenolysis between an internal olefin and ethylene, can potentially yield α-olefins, but the technology is suffering from equilibrium and selectivity limitations. Furthermore, ethenolysis of an internal olefin would result in an olefin with a shorter carbon chain than the starting internal olefin.

1-Hexene can also be produced by means of trimerisation of ethylene. Although this is a well-known method for producing 1-hexene, it has the disadvantage that C₄, C₈ and C₁₀ impurities are also produced.

The inventors of the present invention have now developed a novel process of increasing the carbon chain length of olefinic compounds, including and especially α-olefins. Accordingly, shorter α-olefins such as 1-pentene can be converted to 1-hexene.

Fischer-Tropsch technology produces a large range of hydrocarbon products following an Anderson-Schulz-Flory distribution. This means that more 1-pentene is produced than 1-hexene. The market demand for 1-pentene is small with the result that most of the 1-pentene ends up in a fuel pool, resulting in a fuel alternative value. On the other hand 1-hexene is sold at a much higher value. The same arguments as for 1-pentene hold for heptenes and butenes. It is believed that with the process of the present invention 1-butene, 1-pentene and/or 1-heptene can undergo controlled chain growth reactions to yield 1-hexene and/or 1-octene.

### DISCLOSURE OF THE INVENTION

According to the present invention there is provided a process of increasing the carbon chain length of an olefinic compound comprising the steps of:
providing a starting olefinic compound and subjecting it to hydroformylation to produce an aldehyde and/or alcohol with an increased carbon chain length compared to the starting olefinic compound;
optionally, hydrogenating the aldehyde that forms during the hydroformylation reaction to convert it to an alcohol which has an increased carbon chain length compared to the starting olefinic compound; and
subjecting the alcohol with the increased carbon chain length to dehydration to produce an olefinic compound with an increased carbon chain length compared to the starting olefinic compound.

In this specification the term olefinic compound means an olefin, and a substituted olefin which includes one or more heteroatoms which are neither carbon nor hydrogen.

It will be appreciated that the chain length may be increased by for example lengthening the only carbon chain in the case of unbranched linear compounds, lengthening the longest carbon chain or a branch carbon chain in the case of a branched carbon chain product, or by the formation of a branch carbon chain or the formation of an additional branch carbon chain.

Preferably the process is a process for producing linear unbranched olefins, preferably α-olefins, preferably α-olefins with an even number of carbon atoms, preferably 1-hexene and/or 1-octene.

Preferably the process is one wherein the carbon chain length of an α-olefinic compound with an odd number of carbon atoms is increased by one carbon to an α-olefinic compound with an even number of carbon atoms.

Preferably the starting olefinic compound comprises an olefin, preferably an olefin with a single carbon-carbon double bond. Preferably the starting olefin is an unbranched linear olefin, preferably an α-olefin, and often it will be an α-olefin with an odd number of carbons in the carbon chain, such as 1-pentene and/or 1-heptene.

In one embodiment of the invention 1-pentene may be converted to 1-hexene. Alternatively or additionally 1-heptene may be converted to 1-octene.

In one embodiment of the invention a Fischer-Tropsch derived feed stream containing one or more α-olefins may be used as a source of the starting olefinic compound. Preferably, the feed stream contains a significant concentration of olefins having an odd number of carbon atoms.

It will be appreciated that the process can be used to obtain controlled carbon chain growth of olefinic compounds and the process may be repeated to obtain chain growth of the formed olefinic compound. That is, for example, 1-butene as starting olefin may undergo chain growth by one carbon to be converted to 1-pentene which may then be converted to 1-hexene.

Hydroformylation of olefinic compounds to produce aldehydes and/or alcohols with an increased carbon chain length is well known and can be carried out in many different and known ways. This step and the different options available are accordingly not described in detail in this specification.

It will be appreciated that during hydroformylation of an olefin, hydrogen and carbonyl are added to the carbon atoms across a double bond to yield a compound with an increased carbon chain length compared to the starting olefin. Where the carbon atom of the carbonyl group is bound to hydrogen, an aldehyde is formed. Some aldehydes may, depending on the type of catalyst used, automatically convert to the corresponding alcohol by means of an in situ hydrogeneration reaction. It is believed that in the case of a catalysed hydroformylation reaction, a leaving group (usually in the form of the catalyst or a derivative thereof) will be bound to the carbonyl group. If the leaving group is replaced with H, an aldehyde forms. Alternatively, if the leaving group is replaced with H and hydrogenation takes place, an alcohol forms.

In one embodiment of the invention the hydroformylation step may be carried out by reacting the olefinic compound with CO and H₂ in the presence of a suitable catalyst and under suitable conditions. The catalyst may comprise a suitable Rh catalyst [e.g. Rh(acac)(CO)₂] in combination with triphenyl phosphine), but preferably it comprises a suitable cobalt catalyst e.g. cobalt with the ligand eicosyl phoban.

The reaction may be carried out in a temperature range from 25 to 250° C., preferably from 100 to 200° C. The reaction is preferably carried out at a pressure from 10 to 100 bar_{g}, preferably 60 to 90 bar_{g}.

In a preferred embodiment of the invention the catalyst and reaction conditions are selected to obtain a high selectivity of n-alcohols as reaction product when an α-olefin is used as the starting olefinic compound. Preferably a selectivity of at least 90% is obtained.

In cases where significant amounts of aldehyde is produced during the hydroformylation it is preferred to include a hydrogenation step to convert the aldehyde to an alcohol. Where no significant amounts of aldehyde forms during the hydroformylation, a hydrogenation step may not be required.

The hydrogenation may comprise reacting the aldehyde in a solvent or neat with H₂ in the presence of any suitable hydrogenation catalyst (e.g. Pd—C, Pt—Al₂O₃, Cu/Cr, etc). This is a well known process and is accordingly not described in detail in this specification.

Removal of unwanted products may take place at any stage prior to or after the dehydration process step. Preferably, unwanted alcohols or aldehydes are removed prior to the dehydration step.

Where branched alcohols or aldehydes are produced during the hydroformylation step and optionally the hydrogenation step, and a linear α-olefinic compound is desirable, such branched alcohols or aldehydes may be removed, for example by distillation, before the dehydration step to improve the selectivity to linear olefinic compounds. Unwanted aldehydes can be removed, e.g. by distillation, prior to the hydrogenation step.

A suitable feedstock for hydroformylation may contain a single olefin or may be a mixture of olefin isomers. It will be appreciated that each olefin isomer contained in a mixed olefin feed may form different isomers of aldehydes or alcohols during hydroformylation. For example, 1-pentene may form 1-hexanol or 2-methylpentanol, depending to which carbon atom of the double bond the CO group bonds during hydroformylation. Similarly, the hydroformylation of 1-heptene may yield 1-octanol or 2-methylheptanol and other olefins may yield the corresponding alcohols. The same principle applies where the hydroformylation product is an aldehyde.

It is well known in the art of hydroformylation processes that although extreme endeavours are undertaken to selectively produce a specific isomer as product, a significant concentration of the other isomer also forms. This is the case with all known hydroformylation catalyst types and would occur in hydroformylation reactions irrespective of which catalyst is used.

According to the present invention, the alcohol isomers formed by hydroformylation (and optionally hydrogenation) are dehydrated to yield their corresponding olefinic compound isomers. Where a pure product, for example co-monomer grade α-olefin is the desired product, these olefinic compound isomers must be separated from each other. The olefinic compound isomer mixtures may be purified by distillation processes. However, certain of these olefinic compound isomers have boiling points so close to each other that distillation becomes extremely complex. For example, the boiling points of 1-hexene and 2-methylpentene are 64 and 62° C., respectively. To separate these close boiling compounds by distillation, is extremely capital intensive as distillation columns with a large number of distillation stages are required.

The last column of Table 1 below, shows the boiling points of some of the major olefinic compounds produced by hydroformylation of 1-pentene (and optionally hydrogenation) and dehydration of a C6 alcohol mixture, illustrating the difficulty in separating such compounds due to their close boiling points.

TABLE 1

| | Alcohol compounds in | | Dehydration | |
|---|---|---|---|---|
| | hydroformylation product | | products with | |
| Olefinic compounds in | with boiling points in ° C. | | boiling points in | |
| hydroformylation feed | shown in brackets | | ° C. | |
| | Products from linear | | | |
| Linear olefins | olefins | | | |
| 1-pentene | 1-hexanol | (156.5) | 1-hexene | (64) |
| 2-pentene | 2-methyl-1-pentanol | (148) | 2-methyl-1-pentene | (62) |
| | 2-ethyl-1-butanol | (146) | 2-ethyl-1-butene | (64-65) |
| | Products from branched | | | |
| Branched olefins | olefins | | | |
| 3-methyl-1-butene | 4-methyl-1-pentanol | (160-165) | 4-methyl-1-pentene | (53-54) |
| 2-methyl-2-butene | 2,3-dimethyl-1-butanol | — | 2,3-dimethyl-1- | (56) |
| | | | butene | |
| 2-methyl-1-butene | 3-methyl-1-pentanol | (151-152) | 3-methyl-1-pentene | (54) |

Thus, dehydration products like 1-hexene cannot be separated from 2-methyl-1-pentene and 2-ethyl-1-butene in a fashion which is commercially feasible, to produce a pure 1-hexene product.

The inventors have surprisingly found that the desired olefin can be produced in high purity by removing unwanted compounds prior to the dehydration step, preferably by distillation of the alcohol and/or aldehyde produced by hydroformylation (and optionally hydrogeneration) before dehydration thereof, to produce a high purity olefinic compound (e.g. an α-olefin) with increased carbon chain length when compared to that of the starting olefinic compound. By distillation of the alcohol product, prior to dehydration thereof, close boiling point olefinic compound isomers eg. 2-methyl-1-pentene and 2-ethyl-1-butene are not produced, which enables production of high purity olefins (e.g. α-olefins). The second column in Table 1 above shows that the alcohol isomers prior to dehydration have larger differences in boiling points compared to the olefinic compound isomers.

Thus, according to this invention, desired olefinic compounds (especially α-olefins) can be produced from shorter chain olefinic compounds, in high purities, especially by distillation of the alcohol product prior to dehydration thereof.

Thus, according to this invention, desired α-olefinic compounds (especially α-olefins) can be produced from shorter chain olefinic compounds, in purities of greater than 95% of the desired isomer. More preferably, desired olefinic compounds (especially α-olefins) can be produced from shorter chain olefinic compounds (especially α-olefins), in purities of greater than 98% of the desired isomer.

Any suitable dehydration process may be used to convert the alcohol with the increased carbon chain length, to the olefinic compound. In cases where the alcohol is a n-alcohol (or significant concentrations thereof are present) the dehydration process is preferably controlled to produce α-olefinic compounds.

Many different dehydration processes are known and they are accordingly not discussed in any detail in this specification. Preferably the dehydration is carried out under low acidity conditions and a low acidity catalysts support such as Al₂O₃; SiO₂; TiO₂ or ZrO₂ may be employed to afford a dehydration reaction at temperatures from 200 to 450° C., typically from 250 to 350° C. and at pressures from 0 to 30 bar_{g}, typically 0 to 5 bar_{g}. The catalyst may comprise a gamma-alumina catalyst or a promoted alumina catalyst e.g CaO.Al₂O₃, Ca₂O₃.Al₂O₃.

This invention also relates to a product produced by the process substantially as described hereinabove.

The invention will now be further described by means of the following non-limiting examples.

### EXAMPLE 1

### Hydroformylation of 1-Pentene Using a Cobalt Catalyst

A batch run hydroformylation reaction was performed in a 450 ml Parr autoclave to determine reaction rate and to confirm conversion, selectivity and n:i ratios of aldehydes and alcohols with time on stream.

Firstly, the hydroformylation catalyst was prepared by adding cobalt decanoate, eicosyl phoban (EP) and linear alkyl benzene sulphonate (LABS) (molar ratio Co:EP:LABS=1:3:0.1) into the autoclave under inert conditions. A pentene feedstream (derived from a Fischer-Tropsch synthesis reaction) as described in Table 2 was then added to the autoclave. The concentration of cobalt was kept at 300 ppm. The temperature in the autoclave was then increased in steps of 20° C. until a temperature of 170° C. was reached, where the temperature was maintained for the duration of the experiment. The autoclave was then pressurised with syngas (molar ratio of CO:H₂ being 1:2) to a pressure of 75 bar_{g}. The contents of the reactor was stirred at a speed of 500 rpm for the duration of the reaction. The reaction was allowed to proceed for 48 hours.

TABLE 2

| Compound | Mass % |
|---|---|
| 1- and 2-Butene | 0.42 |
| 1-Pentene | 69.69 |
| 2-Pentene | 2.32 |
| Total linear pentene | 72.01 |
| Branched C₅ olefin | 14.12 |
| Other C₅ olefins (cyclic and dienes) | 0.57 |
| Total other C₅ olefins | 14.69 |
| Total C₅ olefin content | 86.70 |
| Others (paraffins) | 12.88 |
| Total | 100 |

The acidity of the pentene feedstream was 0.005 mg KOH/g feedstream.

Results:

After 48 hours, the reactor product conversion, selectivity and product linearity was found to be as shown in Table 3:

TABLE 3

| C₅ olefin conversion (mass %) | |
|---|---|
| Linear C₅ | 100.0 |
| Total C₅ conversion | 99.9 |
| Reaction Products (mass %) | |
| Aldehydes | 0.1 |
| Total alcohols | 90.4 |
| 1-Hexanol | 71.9 |
| Paraffin | Not determined |
| Heavies | 1.9 |
| Linearities (mass %) | |
| C₆ aldehyde | 100.0 |
| C₆ alcohol | 80.0 |

FIG. 1 gives the analysis of samples taken with time. All the olefins and the aldehydes reacted until there was nothing left after 48 hours. The alcohol concentration after 48 hours was determined to be 90.8% by mass of which 71.9% by mass was hexanol and the heavies were 1.9 (mass %). (The graph in FIG. 1 only illustrates up to 6 hrs reaction time).

[image]

The 1-hexanol produced by this example can be subjected to dehydration to produce 1-hexene.

### EXAMPLE 2

### Hydroformylation of 1-Pentene Using a Rhodium Catalyst

Run 1

A 300 ml Parr autoclave with a 50 ml mounted feed bomb was used. The autoclave was loaded with 100 ml toluene (solvent), ˜0.02415 g Rh (acac) catalyst pre-cursor (˜35 mg/l Rh in 150 ml liquid volume in the autoclave) and 1.028 g TPP (i.e. 1:80 Rh(acac)(CO)₂:TPP ratio) and heated to 80° C. under 5 bar syngas (molar ratio CO:H₂ being 1:1) pressure and 750 rpm stirring speed for ˜45 minutes. While the reaction was heating up, 50 ml 1-pentene was loaded into the feed bomb under 6 bar syngas (connected to the gas reservoir) at room temperature with the valve to the autoclave closed. After 45 minutes, the reaction was initiated by pressuring the system with 6 bar syngas (molar ratio CO:H₂ being 1:1).

Run 2

The experiment was then repeated by using the exact same conditions as described above, but with an impure 1-pentene feedstream of the composition as set out in Table 4.

TABLE 4

| COMPOUND | MASS % |
|---|---|
| non-oxo-reactive olefins (vinylidenes) | 7.3% |
| branched olefins | 6.4% |
| 1-pentene | 61.0% |
| linear internal pentene | 3.3% |
| ethanol | 1.7% |
| acetone | 7.7% |
| branched paraffins | 2.5% |
| n-pentane | 10.1% |

The data analysis over a 2 hour reaction time is shown in Table 5.

TABLE 5

| | | | Mass % | | n:iso ratio | Rate |
|---|---|---|---|---|---|---|
| | Mass % yield of | | selectivity | Yield | of | constant/ |
| | C6 aldehydes | Mass % | to C6 | n-C6 | aldehydes | Time |
| Run | from pentene | conversion | aldehydes | aldehydes | formed | Unit (h⁻¹) |
| Run 2 | 76.36 | 81.13 | 94.1 | 53.1 | 2.3:1 | 14.54 |
| Impure | | | | | | |
| 1- | | | | | | |
| pentene | | | | | | |
| Run 1 | 97.48 | 97.91 | 99.6 | 74.6 | 3.3:1 | 29.97 |
| Pure 1- | | | | | | |
| pentene | | | | | | |

The C₆ aldehydes (especially n-C₆ aldehydes) may be hydrogenated to form C₆ alcohol. The C₆ alcohol may in turn be subjected to dehydration to produce C₆ olefins especially 1-hexene.

### EXAMPLE 3

### Dehydration of the Formed Alcohol

In a vertical pipe reactor 400 mm in length and 25.4 mm inner diameter, a catalyst bed of about 12 grams of gamma-Al₂O₃ was loaded, supported on quartz wool. The reactor was heated to 315° C. and 1-hexanol (98% pure) was fed into the reactor at a LHSV of 5 hr⁻¹ at atmospheric pressure. The reactor product (after removal of water and hexenes) was recycled to the reactor at a mass ratio of feed:recycle of 0.75: 2.0. A total of 94% of the 1-hexanol was converted to alkenes, with a selectivity towards hexenes of 98.6%, of which 97.5% was 1-hexene.

### EXAMPLE 4

### Production of 1-Hexene From 1-Pentene

Step 1: Hydroformylation of 1-Pentene

An impure pentene feedstream derived from a Fischer-Tropsch synthesis reaction containing 70 mass % 1-pentene, trace amounts of internal and branched olefins and the balance C₅ paraffin was subjected to modified cobalt catalysed hydroformylation. The feedstock (6I) along with a stock solution containing: 300 ppm Co(II)octanoate, eicosyl phoban (EP) as ligand and LABS as a surfactant at a ligand:metal:LABS ratio of 3:1:0.1 was added to a 11 l stirred tank reactor (PDU) under inert nitrogen atmosphere. The stirred tank reactor was pressurised to 85 bar with syngas (molar ratio of H₂:CO being 2:1) with the gas fed at a rate of 1 l/min and then heated to 170° C. Approximately 5 l of product was drained per batch. The composition of the crude hydroformylation reaction product is presented in Table 6.

TABLE 6
Hydroformylation product

| Olefin conversion (mass %) | |
|---|---|
| Linear C₅ | 95.6 |
| Total C₅ conversion | 93.7 |
| Product Composition (mass %) | |
| C₆ Aldehydes | 11.5 |
| C₆ Alcohol | 65.0 |
| Heavies | 21.3 |
| n:i mass ratio | |
| C₆ aldehyde | 9.9:1 |
| C₆ alcohol | 16.5:1 |
| Product Linearities (mass %) | |
| C₆ aldehyde | 78.8 |
| C₆ alcohol | 85.9 |

Step 2: Purification of Hydroformylation Product

After the crude hydroformylation product was produced in the PDU, the cobalt catalyst was removed on a short path distillation (SPD) unit. The hydroformylation product and subsequent bottoms were allowed to pass through the SPD unit four times in order to remove the wide range of products namely paraffins, olefins, aldehydes, alcohols and heavies. A total of 32.0 kg (39.2 l) of overhead product was recovered (90% recovery). The conditions for each pass through the SPD unit are given in Table 7 and the product selectivity after distillation is given in Table 8.

TABLE 7
Operating conditions used in the SPD unit

| Pass # | Temperature | Pressure |
|---|---|---|
| 1^{st} | 160° C. | Atmospheric |
| 2^{nd} | 175° C. | Atmospheric |
| 3^{rd} | 130° C. | 50 mbar_{g} |
| 4^{th} | 175° C. | 1 mbar_{g} |

TABLE 8
Product selectivity of oxo product before and after distillation

| | SPD feed | SPD overheads |
|---|---|---|
| C₆ product composition (mass %) | | |
| Aldehydes | 11.5 | 7.0 |
| Alcohol | 65.0 | 68.3 |
| Heavies | 21.3 | — |
| n:i mass ratios | | |
| C₆ aldehyde | 9.9 | 8.5 |
| C₆ alcohol | 16.5 | 15.7 |
| Linearities (mass %) | | |
| C₆ aldehyde | 78.8 | 77.8 |
| C₆ alcohol | 85.9 | 85.5 |

The wet chemical analysis of the hydroformylation product after the distillation step showed a water content (mass %) of 0.45, acidity of 0.54 mgKOH/g, and a Br number (g Br₂/100 g) of 6.25.

Step 3: Hydrogenation of Hydroformylation Product

A total volume of 650 ml of pelletised Cu/Cr 1152T catalyst was thoroughly mixed with 650 ml of carborundum (>1 mm) before being loaded into a 1 litre, 25 mm diameter hydrogenation reactor on top of 20 ml of glass balls. The loading was performed in small batches under a nitrogen atmosphere. Glass balls (20 ml) were also loaded on top of the catalyst bed.

The catalyst was reduced under hydrogen atmosphere, whereafter the reactor temperature was decreased to 140° C. under a hydrogen flow rate of 0.179 m³ₙ/h, i.e. 0.275 m³ₙ/h/1 litre catalyst hour, while the reactor pressure was being increased to 60 bar at a rate of 3 bar/min. In order to wet the catalyst surface, hexanol was fed to the reactor at a rate of 0.16 kg/h once stable process conditions had been reached. The product vessels were drained after an hour and the product was added to the feed vessel. The feed vessel as well as the product vessels i.e. the vessels used to store the product, was kept under a nitrogen atmosphere to prevent degrading of the product and evaporation of the light materials.

The reactor temperature was gradually increased to 170° C. under a hydrogen flow rate of 0.179 m³ₙ/h, whilst constantly feeding hexanol at a rate of 0.16 kg/h. Upon reaching the desired reactor temperature of 170° C., the product vessels were drained and the hydroformylation product of step 2 was added to the feed vessel. This feed was processed at a reactor temperature of 170° C., a reactor pressure of 60 bar, a hydrogen flow rate of 0.179 m³ₙ/h and a hexanol flow rate of 0.16 kg/h. Once all the feed had been processed, the HPLC pump was switched off and the reactor temperature was decreased to 140° C.

Table 9 provides the results of all the analyses performed on the feed sample as well as the hydrogenation product samples.

TABLE 9
Analytical results of the feed and the product samples.

| | Feed | Hydrogenation |
|---|---|---|
| Analyses | composition | product |
| Acid number (mg. KOH/g.) | 0.42 | 0.01 |
| Carbonyls (ppm = CO) | 32278 | 50 |
| Esters (mg. KOH/g.) | 11.2 | 3.6 |
| Bromine (g BR/100 g.) | 0.89 | 0.02 |
| Aldehydes | 1.55 | 0.06 |

Step 4: Purification of 1-Hexanol

The hydrogenated product of step 3 contained a high concentration of 1-hexanol and the hydrogenated product was then distilled to remove the light components and the branched alcohols, using a 6 m long column having 48 theoretical stages. Distillation produced a hexanol cut consisting of 99.6% 1-hexanol, 0.20% 3-methyl-1-pentanol, 0.08% 4-methyl-1-pentanol and 0.12% of other minor components.

Step 5: Dehydration of 1-Hexanol

The purified hexanol of step 4 was subjected to dehydration carried out over a γ-alumina catalyst (produced by SASOL Chemie, Pural KR1). The reaction conditions employed were 290° C., WHSV=8 h⁻¹ and atmospheric pressure. The alumina was pretreated at the reaction temperature overnight under a flow of nitrogen. An analysis of the product obtained by dehydration is shown in Table 10, below.

TABLE 10
Analysis of product obtained by the dehydration of
1-hexanol at 76.5% conversion and 7 hours
on-stream. Results are expressed on dry-basis.

| Compound | Mass % |
|---|---|
| 3-methyl-1-pentene | 0.01 |
| 1-hexene | 54.21 |
| n-hexane | 0.12 |
| trans-3-hexene | 0.02 |
| cis-3-hexene | 0.03 |
| trans-2-hexene | 0.39 |
| cis-2-hexene | 1.26 |
| trans-3-methyl-2-pentene | 0.15 |
| 1-hexanol | 26.57 |
| dihexyl ether | 17.00 |
| minor components | 0.24 |
| Total | 100.00 |

NOTES
1. The minor components were:
(i) those eluting before the hexenes(0.04%), by GC analysis
(ii) those eluting after the C₆ alkanes and alkenes and before 1-hexanol (total of 0.13%)
(iii) those eluting between 1-hexanol and dihexyl ether (total of 0.07%)
2. At this conversion level, 13.7 g of water was produced, per 100 g of total product.

Step 6: Purification of Dehydration Product

The dehydration mixture (see Table 10) was first distilled to separate the lights and the C₆ fraction from the unreacted hexanol, the dihexyl ether and the heavies. This preliminary distillation produced a cut containing 97% 1-hexene. This was then followed by distillation of the lights and the C₆ fraction in a 6 m column having 48 theoretical stages, to produce a 1-hexene product of >99% purity.

The composition of the final 1-hexene product is listed in Table 11.

TABLE 11
Analysis of 1-hexene product

| Compound | Mass % |
|---|---|
| 4-methyl-1-pentene | 0.03 |
| 3-methyl-1-pentene | 0.11 |
| 1-hexene | 99.84 |
| n-hexane | 0.01 |
| other minor | 0.01 |
| components | |
| Total | 100.00 |

### EXAMPLE 5

### Production of 1Octene From 1-Heptene

Step 1: Hydroformylation of 1-Heptene

An impure feedstock containing 75 mass % 1-heptene, trace amounts of internal and branched olefins and the balance C₇ paraffin was subjected to hydroformylation conditions. To 360 ml of this feedstock was added in a 600 ml Parr reactor, 1000 ppm Co catalyst with eicosyl phoban (EP) as ligand at a ligand:metal ratio of 4:1. The cobalt stock solution, ligand and olefin feed were mixed in the Parr reactor, under inert atmosphere. After flushing the reactor with argon, the reactor was heated up to the reaction temperature of 170° C., under atmospheric pressure. The reactor temperature was allowed to stabilise at 170° C. before pressurising to 75 bar with syngas of a molar ratio H₂:CO of 2:1.

The reaction was run to exhaustion over 24 hours. The activity was monitored by the rate derived from gas consumption and selectivity derived from end of run analysis. Mass flow meter was used to measure gas consumption and a GC for product analysis. The reaction was then repeated.

Table 12 contains an analysis of the product obtained from the hydroformylation run.

TABLE 12
Hydroformylation product

| | Run1 | Run 2 |
|---|---|---|
| C₈ Aldehyde | | |
| n:iso (mass ratio) | 24.93:1 | 22.26:1 |
| Linearity (mass %) | 83.47 | 86.18 |
| Total aldehyde formed | 5.66 | 7.87 |
| (mass %)* | | |
| C₈ Alcohol | | |
| n:iso (mass ratio) | 19.20:1 | 20.36:1 |
| Linearity (mass %) | 88.18 | 86.66 |
| Octanol (mass %) | 74.88 | 74.84 |
| Total Alcohol formed | 92.78 | 90.95 |
| (mass %)* | | |
| Olefin Conversion | 99.90 | 99.90 |
| (mass %) | | |
| k-values (h⁻¹) | 0.17 | 0.12 |

*In total product as determined by GC analysis.

Step 2: Purification of Hydroformylation Product

Hydroformylation produces “heavy” by-products such as aldol products, acetals and esters. The desired products and the lights comprising paraffin and trace amounts of unreacted olefins were recovered from the “heavy” by-products using short path distillation (SPD). This separation helped to remove unwanted by-products that could have complicated the hydrogenation step. Analysis of the distilled product was done on a GC to ensure that product distribution has not been negatively affected (Table 13).

TABLE 13
Product distribution after SPD

| | After SPD |
|---|---|
| C₈ Aldehyde | |
| n:iso (mass ratio) | 18.3:1 |
| Linearity (mass %) | 84.85 |
| Total C₈ Aldehyde* | 5.51 |
| (mass %) | |
| C₈ Alcohol | |
| n:iso (mass ratio) | 18.54:1 |
| Linearity (mass %) | 88.09 |
| Octanol (mass %) | 76.09 |
| Total C₈ Alcohol* | 92.42 |
| (mass %) | |

*In total product as determined by GC analysis.

Step 3: Hydrogenation of Hydroformylation Product

To a 300-ml reactor was added heptane (130-ml), 20 ml of the purified hydroformylation product of step 2 and 0.15 g of crushed Cu/Cr 1152T catalyst. The reactor temperature was increased to 165° C., before the pressure was increased to 65 bar(g). GC analysis showed that aldehyde content was reduced to 0.1 mass %. The reaction was then scaled up with a large product sample being hydrogenated in a 600-ml reactor. Analysis of the resultant product yielded total aldehyde content of less than 0.16% content.

Step 4: Dehydration of 1-Octanol

The 1-octanol feed obtained from step 3, and which contained approximately 12% of branched octanols, was subjected to dehydration. The experimental procedure employed during dehydration, was as described in example 4. The results presented here are for a total of 8 hours on-stream. The conditions employed were a reaction temperature of 280° C. and an MHSV of 10 h⁻¹. The results obtained are shown in Table 14 and FIG. 2.

[image]

TABLE 14
Results from the dehydration of the
1-octanol feed with time-on-stream

| | | | | | Corrected |
|---|---|---|---|---|---|
| | | mass % | mass % | mass % | mass % |
| TOS | mass % | Selectivity | Selectivity to | 1-octene | purity of |
| (h⁻¹) | Conversion | to octenes | dioctyl ethers | Purity | 1-octene |
| 1 | 73.2 | 56.0 | 17.9 | 85.0 | 97.4 |
| 2 | 69.0 | 51.4 | 22.5 | 85.3 | 97.6 |
| 3 | 66.3 | 45.2 | 27.4 | 85.3 | 97.9 |
| 4 | 61.9 | 40.5 | 31.0 | 85.4 | 98.1 |
| 5 | 60.8 | 40.2 | 30.8 | 85.4 | 98.2 |
| 6 | 60.1 | 37.6 | 33.1 | 85.4 | 98.2 |
| 7 | 59.8 | 36.5 | 33.9 | 85.4 | 98.3 |
| 8 | 59.4 | 36.4 | 33.9 | 85.5 | 98.3 |

Due to the presence of branched octanols (approx. 12%) in the hydroformylation product, the observed purity of 1-octene fraction is only about 85%.

However, branched alcohols may optionally be removed from the hydroformylation product before dehydration. The “corrected” purities included in Table 14 represents expected values where a hydroformylation product which is free of branched alcohols, is dehydrated. Therefore, with proper purification of the 1-octanol feed it could be possible to produce 1-octene fractions with purities higher than 95% at high conversions of the alcohol.

1. A process of increasing the carbon chain length of an olefinic compound comprising the steps of:
providing a starting olefinic compound and subjecting it to hydroformylation to produce an aldehyde and/or alcohol with an increased carbon chain length compared to the starting olefinic compound;
optionally, hydrogenating the aldehyde that forms during the hydroformylation reaction to convert it to an alcohol which has an increased carbon chain length compared to the starting olefinic compound; and
subjecting the alcohol with the increased carbon chain length to dehydration to produce an olefinic compound with an increased carbon chain length compared to the starting olefinic compound.

2. The process of claim 1 wherein the carbon chain length of an olefinic compound with an odd number of carbon atoms is increased by one carbon to an α-olefinic compound with an even number of carbon atoms.

3. The process of claim 2 wherein 1-pentene is converted to 1-hexene.

4. The process of claim 2 wherein 1-heptene is converted to 1-octene.

5. The process of claim 1 wherein the starting olefinic compound comprises an unbranched linear α-olefin with a single carbon-carbon double bond.

6. The process of claim 1 wherein a Fischer-Tropsch derived feed stream containing one or more olefins is used as the starting olefinic compound.

7. The process of any one of the preceding claims wherein the hydroformylation is carried out by reacting the olefinic compound with CO and H₂ in the presence of a suitable catalyst.

8. The process of any one of the preceding claims wherein significant amounts of aldehyde is produced during hydroformylation, the process including the step of hydrogenating the aldehyde to convert it to an alcohol which has an increased carbon chain length compared to the starting olefinic compound.

9. The process of any one of the preceding claims which includes the removal of unwanted products before or after the dehydration step.

10. The process of claim 9 where unwanted alcohols or aldehydes are removed prior to the dehydration step.

11. An olefinic compound produced by the process of any one of the preceding claims.
